(19) United States
(12) Patent Application Publication
(10) Pub. No.: US 2021/0052632 A1
(43) Pub. Date: Feb. 25, 2021

(54) SELECTIVE PARP1 INHIBITORS TO TREAT CANCER

(71) Applicant: Cambridge Enterprise Limited, Cambridge, Cambridgeshire (GB)

(72) Inventors: Melinda DUER, Cambridge, Cambridgeshire (GB); David REID, Cambridge, Cambridgeshire (GB); Uliana BASHTANOVA, Cambridge, Cambridgeshire (GB)

(21) Appl. No.: 16/959,519

(22) PCT Filed: Jan. 16, 2019
(86) PCT No.: PCT/GB2019/050110

§ 371 Date: Jul. 1, 2020

(30) Foreign Application Priority Data
Jan. 17, 2018 (GB) 1800733.6

(51) Int. Cl. A61K 31/7135; A61K 31/28

(52) U.S. Cl. CPC A61K 31/7135; A61K 45/06; A61K 31/28

(57) ABSTRACT

The disclosure provides a selective inhibitor of DNA-binding to poly (ADP-ribose) polymerase 1 (PARP1), or a pharmaceutically acceptable salt or solvate thereof, for use in treating, ameliorating or preventing cancer. The treatment may be given to a subject suffering from or at risk of osteoporosis or a subject requiring a long-term therapy.

The invention relates to cancer, and in particular to novel compositions, therapies and methods for treating, preventing or ameliorating cancer.

Poly (ADP-ribose) polymerase 1 (PARP1) acts in the cell nucleus to repair both single-strand DNA breaks (SSBs) and double strand breaks (DSBs), inclusive homologous recombination (HR) and non-homologous end joining (NHEJ) repair. This PARP1-mediated DNA repair mechanism provides an opportunity to kill cancerous cells, which are either naturally defective in BRCA genes or affected by DNA-damaging antitumoral drug/ionising radiation. This is because BRCA1 and BRCA2 are proteins involved in important DNA repair mechanisms. If either or both of these proteins are defective for any reason, cells rely much more strongly on PARP-mediated DNA repair pathways. PARP1 inhibition in such cases induces so-called “synthetic lethality” in cancer cells. This is the basis for the drug approvals of the PARP inhibitors olaparib (LYNPARZA™), rucaparib (RUBRACA™), niraparib (ZEJULA™) and talazoparib (TALZENNA™).

PARP1 binds to damaged DNA through zinc finger domains, an event that causes a series of allosteric changes in the structure of PARP1 that significantly activates its catalytic function. The NAD+ mediated PARylation process occurs at the catalytic PARP domain, catalysing poly(ADP-ribosyl)ation of PARP1 itself (an automodification reaction) and other various nuclear proteins including histones (heteromodification reaction) (see De Vos et al. “The diverse roles and clinical relevance of PARPs in DNA damage repair: Current state of the art”, Biochemical Pharmacology 84 (2012) 137-146), that signals and attracts repair proteins to the DNA lesion sites. The autoPARylation of PARP1 changes its conformation and this allows the PARP1 to subsequently release from the DNA binding site. Once released, other molecules then strip the PARylation modifications from PARP1, such that it can then bind to another DNA lesion site and repeat the repair process (Lord et al. “PARP inhibitors: Synthetic lethality in the clinic” Science 17 Mar. 2017: Vol. 355, Issue 6330).

Existing PARP1 inhibitors are thought to bind to the catalytic domain of PARP1, including the re-structured catalytic domain of PARP1 bound to a DNA lesion site via its zinc finger domains. The inhibitor prevents PARylation occurring at the catalytic domain by inhibiting binding of the enzyme's substrate (β-NAD). In the case of SSB/DSB repair, this results in DNA-bound PARP1 not being PARylated, and the other proteins involved in DNA repair are therefore not attracted to the SSB/DSB site, so repair does not occur, and PARP1 is “trapped” at the DNA lesion site, as it cannot disassociate from DNA unless it is PARylated (Lord et al supra).

PARP1 has roles that are independent of DNA damage. For instance, acetylation of PARP1 under cellular stress conditions activates its enzymatic activity even in the absence of DNA (“SIRT1 Promotes Cell Survival under Stress by Deacetylation-Dependent Deactivation of Poly(ADP-Ribose) Polymerase 1,” Rajamohan et al, Molec. Cell Biol. 2009; 29(15): 4116-4129). There is significant evidence that PARP1 is involved in cellular response to oxidative stress, independent of DNA damage, relevant to non-cancerous cells, reviewed in “On PAR with PARP: cellular stress signaling through poly (ADP-ribose) and PARP-1,” Luo and Kraus, Genes and Development 2012; 26: 417-432 for instance. Moreover, PARP1 has roles in cell metabolic regulation and metabolic activity, again relevant to non-cancerous cells (“The role of PARP-1 and PARP-2 enzymes in metabolic regulation and disease,” Bai and Cant, Cell Metabolism, 2012; 16(3): 290-295; Brunyanszki et al. “Mitochondrial poly(ADP-ribose)polymerase: The Wizard of Oz at work.” Free Radical Biology and Medicine 100 (2016) 257-270). PARP1 with an inhibitor bound to its catalytic domain cannot undertake any other roles, including those just described which are crucial for functioning of non-cancerous cells (Morales et al, “Review of Poly (ADP-ribose) Polymerase (PARP) Mechanisms of Action and Rationale for Targeting in Cancer and Other Diseases”. Crit Rev Eukaryot Gene Expr. 2014; 24(1): 15-28). Accordingly, it would be advantageous to be able to inhibit the DNA repair mechanism of PARP1, while allowing it to continue its other roles.

Similarly other PARP enzymes relevant in DNA repair, namely PARP2 and PARP3 also have roles outside of DNA repair, such as metabolic function and cellular stress response (“Identification of candidate substrates for poly(ADP-ribose) polymerase-2 (PARP2) in the absence of DNA damage using high-density protein microarrays,” Troiani et al, FEBS J. 2011; 278(19):3676-3687; “A systematic analysis of the PARP protein family identifies new functions critical for cell physiology,” Vyas et al, Nature Comm. 2013; 4:2240; “TRPM2 channel opening in response to oxidative stress is dependent on activation of poly(ADP-ribose) polymerase,” British J. Pharmacol. 2004; 143(1):186-192; “Biology of Poly(ADP-Ribose) Polymerases: The Factotums of Cell Maintenance,” Bai, Molec. Cell 2015; 58(6): 947-958; “A fast signal-induced activation of poly(ADP-ribose) polymerase: A novel downstream target of phospholipase C,” Homburg et al, J. Cell Biol. 2000; 150(2):293-307;) and mitochondrial function (“Poly(ADP-ribose) polymerases as modulators of mitochondrial activity,” Bai et al, Trends Endocrin. Metabol. 2015; 26(2): 75-83). Neither PARP2 nor PARP3 can enable DNA repair if PARP1 is not involved, thus their inhibition within the BRCA concept of ‘synthetic lethality’ is unnecessary. Moreover, their inhibition can be damaging for the other essential cell functions listed above. In particular, PARP2 is involved in cellular metabolic regulation and metabolic activity, calcium signalling and calcification, and apoptosis. We describe how inhibiting PARP2 causes osteoblast function loss. Inhibiting PARP2 is therefore a significant risk factor for osteoporosis, a well-known complication of several cancer types including breast cancer and prostate cancer, and a likely complication of long-term use e.g. in a maintenance treatment setting.

Thus it may be important in cancer treatment using PARP inhibition to selectively inhibit DNA-dependent PARP1 activity so as not to interfere with normal possibly protective PARP activity in non-cancerous cells. Alternatively, or additionally, if a cancer develops drug-resistance to PARP inhibitors targeting the catalytic site of PARP enzymes a second PARP inhibitor that has a different mechanism of action in the treatment protocol could be advantageous. Such resistance mechanisms can include phosphorylation of PARP1 by c-Met, elevated expression of ABCB1(MDR1)-the drug efflux pump, activation of mTOR pathway via S6 phosphorylation and other yet to be discovered mechanisms of resistance, which does not include impaired trapping of PARP1 (reviewed in “Reverse the resistance to PARP inhibitiors”, Kim et al., Int. J. Biol. Sci. 2017; 13(2): 198-208).

The present invention arises from the inventors' work in attempting to overcome the problems associated with the prior art.

In accordance with a first aspect of the invention, there is provided a selective inhibitor of DNA-binding to poly (ADP-ribose) polymerase 1 (PARP1), or a pharmaceutically acceptable salt or solvate thereof, for use in treating, ameliorating or preventing cancer in a subject suffering from or at risk of osteoporosis or a subject requiring a long-term therapy.

In a second aspect, there is provided a method of treating, preventing or ameliorating cancer in a subject, the method comprising administering to a subject in need of such treatment, a therapeutically effective amount of a selective inhibitor of DNA-binding to poly (ADP-ribose) polymerase 1 (PARP1), or a pharmaceutically acceptable salt or solvate thereof, wherein the subject is suffering from or at risk of osteoporosis or requires a long-term therapy.

Advantageously, the selective inhibition of DNA-binding to PARP1 prevents SSBs from being repaired. Accordingly, the synthetic lethality mechanism aimed at killing cancer cells is preserved. However, the PARP1 will be available to undertake its other essential cellular roles that do not require DNA-binding to PARP1 in non-cancerous cells in the rest of the body.

It may be understood that a selective inhibitor of DNA-binding to PARP1 does not inhibit the other functions of PARP1 besides DNA-binding. The other functions of PARP1 may comprise PARP1's role in a cellular response to oxidative stress independent of DNA damage and/or PARP1's role in cell metabolic regulation and metabolic activity, calcium signalling and calcification, and apoptosis. The inhibitor may not inhibit or block the NAD+ binding site of PARP1. Preferably, the inhibitor is an inhibitor of the zinc finger of PARP1.

The subject may be considered to be at risk of osteoporosis if the subject is a post-menopausal woman, a woman who has had a hysterectomy before the age of 45, a woman who has suffered from absent periods for more than 6 months as a result of over exercising or too much dieting or a man suffering from hypogonadism. The post-menopausal woman may have undergone an early menopause, i.e. she may have undergone the menopause before the age of 45.

Alternatively, or additionally, the subject may be considered to be at risk of osteoporosis if the subject suffers from rheumatoid arthritis.

The cancer may be a solid tumour or solid cancer. The cancer may be blood cancer, bowel cancer, brain cancer, breast cancer, cervical cancer, endometrial cancer, gastric cancer, liver cancer, lung cancer, ovarian cancer, pancreatic cancer, prostate cancer or skin cancer. The blood cancer may be myeloma. The bowel cancer may be colon cancer or rectal cancer. The brain cancer may be a glioma or a glioblastoma. The breast cancer may be a BRCA positive breast cancer. The breast cancer may be a HER2 positive breast cancer or HER2 negative breast cancer. The liver cancer may be hepatocellular carcinoma. The lung cancer may be non-small cell lung cancer or small cell lung cancer. The skin cancer may be a melanoma.

Some types of cancer increase the risk of osteoporosis. Accordingly, the subject may be considered to be at risk of osteoporosis if the cancer is breast cancer, prostate cancer, myeloma or cervical cancer.

A long-term therapy may be maintenance therapy. Accordingly, the subject may have a cancer in remission.

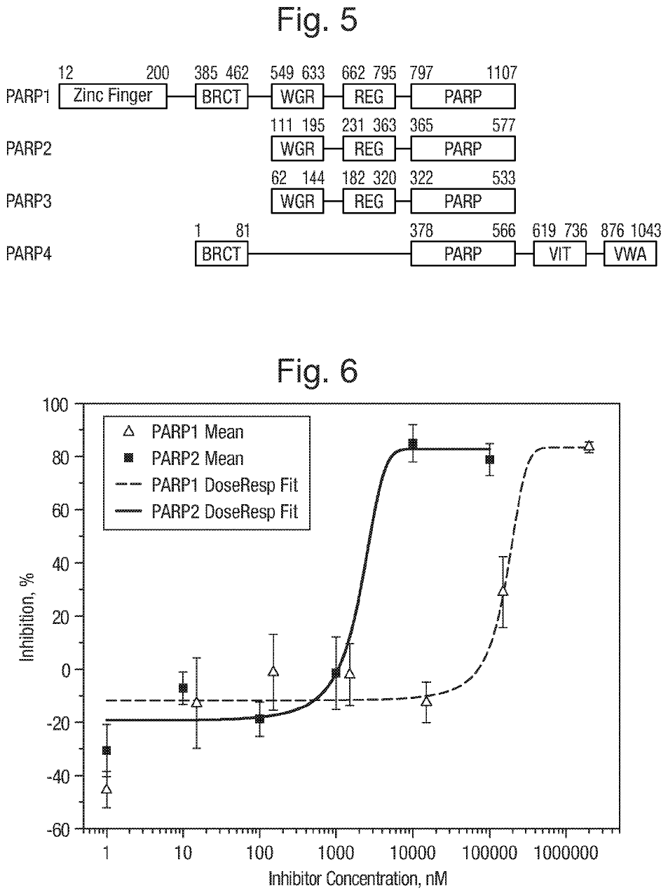

It may be appreciated that the zinc finger domains of PARP1 are involved with DNA binding, and so the inhibitor prevents, reduces or inhibits the ability of PARP1 to bind to DNA. As shown in FIG. 5, the inventors realised that only PARP1 has zinc finger domains in its structure, whereas the other PARP enzymes thought to be involved in DNA repair, PARP2 and PARP3 do not. PARP2 and PARP3 also have many other cellular roles in non-cancerous cells, not involving DNA repair. Hence, preferably, the inhibitor is not an inhibitor of PARP2 and/or PARP3.

Preferably, the inhibitor is a gold complex, and more preferably a gold (I) complex. Preferably, the inhibitor is a polymeric water-soluble complex. Preferably, the inhibitor is a compound of Formula I, Formula II, Formula III, Formula IV or Formula V:

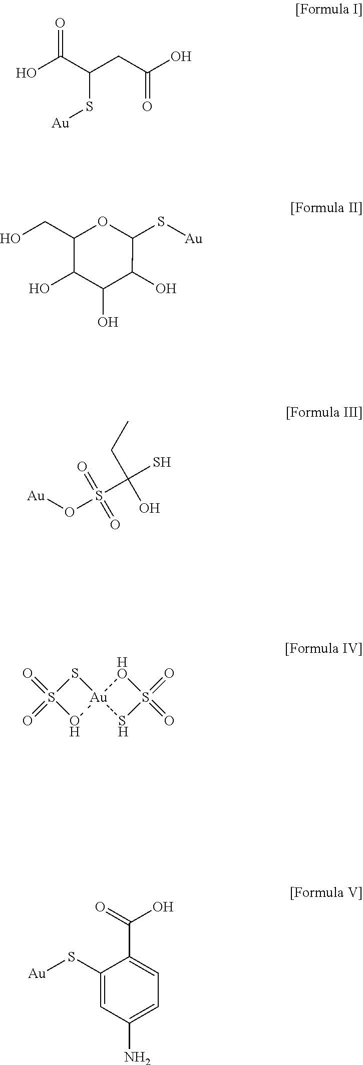

or a pharmaceutically acceptable salt and/or solvate thereof. It may be appreciated that atoms in the above compounds may be replaced with isotopes thereof, and the compound will still fall within the scope of the formula. For instance, a hydrogen in one of the above structures could be replaced with a deuterium, and such a compound would fall within the scope of the relevant formula.

Accordingly, the inhibitor may comprise aurothiomalate, aurothioglucose, gold thiopropanolsulphonate, gold thiosulphate or gold 4-amino-2-mercaptobenzoic acid or a pharmaceutically acceptable salt or solvate thereof.

More preferably, the compound is a compound of Formula I or Formula II. Preferably, the compound of Formula II is a compound of Formula IIa:

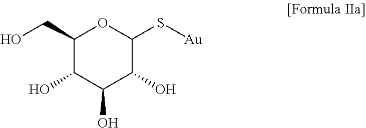

or a pharmaceutically acceptable salt and/or solvate thereof.

Accordingly, the inhibitor may be an aurothiomalate, aurothioglucose or a pharmaceutically acceptable salt or solvate thereof.

Pharmaceutically acceptable salts include any salt of a selective inhibitor of DNA-binding to PARP1 provided herein which retains its biological properties and which is not toxic or otherwise undesirable for pharmaceutical use. The pharmaceutically acceptable salt may be derived from a variety of organic and inorganic counter-ions well known in the art.

The pharmaceutically acceptable salt may comprise an acid addition salt formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids. Alternatively, the pharmaceutically acceptable salt may comprise a base addition salt formed when an acidic proton present in the parent compound is either replaced by a metal ion, e.g., an alkali metal ion, an alkaline earth ion, an aluminium ion, alkali metal or alkaline earth metal hydroxides, such as sodium, potassium, calcium, magnesium, aluminium, lithium, zinc, and barium hydroxide, or coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Accordingly, the salt may comprise a group I or a group II metal salt, i.e. an alkali metal salt or an alkaline earth metal salt. Accordingly, the salt may comprise a lithium salt, a sodium salt, a potassium salt, a beryllium salt, a magnesium salt or a calcium salt.

Accordingly, the aurothiomalate may comprise sodium aurothiomalate, potassium aurothiomalate or calcium aurothiomalate. Preferably, the aurothiomalate comprises sodium aurothiomalate.

Accordingly, the inhibitor may be a compound of Formula Ia:

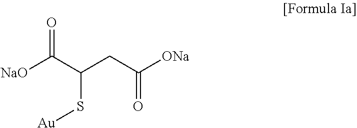

or a pharmaceutically acceptable solvate thereof.

A pharmaceutically acceptable solvate refers to a selective inhibitor of DNA-binding to PARP1, or a salt thereof, that further includes a stoichiometric or non-stoichiometric amount of solvent bound by non-covalent intermolecular forces. Where the solvent is water, the solvate is a hydrate.

It will be appreciated that the inhibitor described herein, or a pharmaceutically acceptable salt or solvate thereof, may be used in a medicament which may be used in a monotherapy (i.e. use of the inhibitor alone), for treating, ameliorating, or preventing cancer. Alternatively, the inhibitor or a pharmaceutically acceptable salt or solvate thereof may be used as an adjunct to, or in combination with, known therapies for treating, ameliorating, or preventing cancer. For example, the inhibitor may be used in combination with a drug that damages DNA. Accordingly, the inhibitor may be used in combination with an ataxia-telangiectasia mutated and rad3-related protein kinase (ATR) inhibitor, a checkpoint inhibitor, a vascular endothelial growth factor (VEGF) inhibitor or a wee1 inhibitor. The checkpoint inhibitor may be a programmed cell death protein 1 (PD-1) inhibitor, a programmed death-ligand 1 (PD-L1) inhibitor or a cytotoxic T-lymphocyte-associated protein 4 (CTLA-4) inhibitor.

Alternatively, or additionally, the inhibitor may be used in combination with ionising radiation that damages DNA.

The inhibitor may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the inhibitor described herein may be used in a number of ways. Compositions comprising the inhibitor of the invention may be administered by inhalation (e.g. intranasally). Compositions may also be formulated for topical use. For instance, creams or ointments may be applied to the skin.

The inhibitor according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the inhibitor used according to the invention is required and which would normally require frequent administration (e.g. at least daily injection).

The inhibitor and compositions according to the invention may be administered to a subject by injection into the blood stream or directly into a site requiring treatment, for example into a cancerous tumour or into the blood stream adjacent thereto. Injections may be intravenous (bolus or infusion) or subcutaneous (bolus or infusion), intradermal (bolus or infusion) or intramuscular (bolus or infusion).

In a preferred embodiment, the inhibitor is administered orally. Accordingly, the inhibitor may be contained within a composition that may, for example, be ingested orally in the form of a tablet, capsule or liquid.

It will be appreciated that the amount of the inhibitor that is required is determined by its biological activity and bioavailability, which in turn depends on the mode of administration, the physiochemical properties of the inhibitor, and whether it is being used as a monotherapy, or in a combined therapy. The frequency of administration will also be influenced by the half-life of the inhibitor within the subject being treated. Optimal dosages to be administered may be determined by those skilled in the art, and will vary with the particular inhibitor in use, the strength of the pharmaceutical composition, the mode of administration, and the advancement of the cancer. Additional factors depending on the particular subject being treated will result in a need to adjust dosages, including subject age, weight, gender, diet, and time of administration.

The inhibitor may be administered before, during or after onset of the cancer to be treated. Daily doses may be given as a single administration. However, preferably, the inhibitor is given two or more times during a day, and most preferably twice a day.

Generally, a daily dose of between 0.01 μg/kg of body weight and 500 mg/kg of body weight of the inhibitor according to the invention may be used for treating, ameliorating, or preventing cancer. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

A patient receiving treatment may take a first dose upon waking and then a second dose in the evening (if on a two dose regime) or at 3- or 4-hourly intervals thereafter. Alternatively, a slow release device may be used to provide optimal doses of the inhibitor according to the invention to a patient without the need to administer repeated doses.

Known procedures, such as those conventionally employed by the pharmaceutical industry (e.g. in vivo experimentation, clinical trials, etc.), may be used to form specific formulations comprising the inhibitor according to the invention and precise therapeutic regimes (such as daily doses of the inhibitor and the frequency of administration). The inventors believe that they are the first to describe a pharmaceutical composition for treating cancer, based on the use of the inhibitor of the invention.

Hence, in a third aspect of the invention, there is provided a pharmaceutical composition for treating cancer in a subject suffering from or at risk of osteoporosis or a subject requiring a long-term therapy, the composition comprising an inhibitor of the first aspect, or a pharmaceutically acceptable salt or solvate thereof, and a pharmaceutically acceptable vehicle.

The pharmaceutical composition can be used in the therapeutic amelioration, prevention or treatment in a subject of cancer.

The pharmaceutical composition may further comprise a drug that damages DNA. The DNA damaging drug may an ataxia-telangiectasia mutated and rad3-related protein kinase (ATR) inhibitor, a checkpoint inhibitor, a vascular endothelial growth factor (VEGF) inhibitor or a wee1 inhibitor. The checkpoint inhibitor may be a programmed cell death protein 1 (PD-1) inhibitor, a programmed death-ligand 1 (PD-L1) inhibitor or a cytotoxic T-lymphocyte-associated protein 4 (CTLA-4) inhibitor.

The invention also provides, in a fourth aspect, a process for making the composition according to the third aspect, the process comprising contacting a therapeutically effective amount of an inhibitor of the first aspect, or a pharmaceutically acceptable salt or solvate thereof, and a pharmaceutically acceptable vehicle.

A “subject” may be a vertebrate, mammal, or domestic animal. Hence, the inhibitor, compositions and medicaments according to the invention may be used to treat any mammal, for example livestock (e.g. a horse), pets, or may be used in other veterinary applications. Most preferably, however, the subject is a human being.

A “therapeutically effective amount” of the inhibitor is any amount which, when administered to a subject, is the amount of drug that is needed to treat the cancer.

For example, the therapeutically effective amount of the inhibitor used may be from about 0.01 mg to about 800 mg, and preferably from about 0.01 mg to about 500 mg. It is preferred that the amount of the inhibitor is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the inhibitor) according to the invention. In tablets, the inhibitor may be mixed with a vehicle having the necessary compression properties in suitable proportions and compacted in the shape and size desired. The powders and tablets preferably contain up to 99% of the inhibitor. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The inhibitor according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The inhibitor may be prepared as a sterile solid composition that may be dissolved or suspended at the time of administration using sterile water, saline, or other appropriate sterile injectable medium.

The inhibitor and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The inhibitor used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

In accordance with a further aspect of the invention, there is provided a selective inhibitor of DNA-binding to poly (ADP-ribose) polymerase 1 (PARP1), or a pharmaceutically acceptable salt or solvate thereof, for use in treating, ameliorating or preventing cancer.

In a still further aspect, there is provided a method of treating, preventing or ameliorating cancer in a subject, the method comprising administering to a subject in need of such treatment, a therapeutically effective amount of a selective inhibitor of DNA-binding to poly (ADP-ribose) polymerase 1 (PARP1), or a pharmaceutically acceptable salt or solvate thereof.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

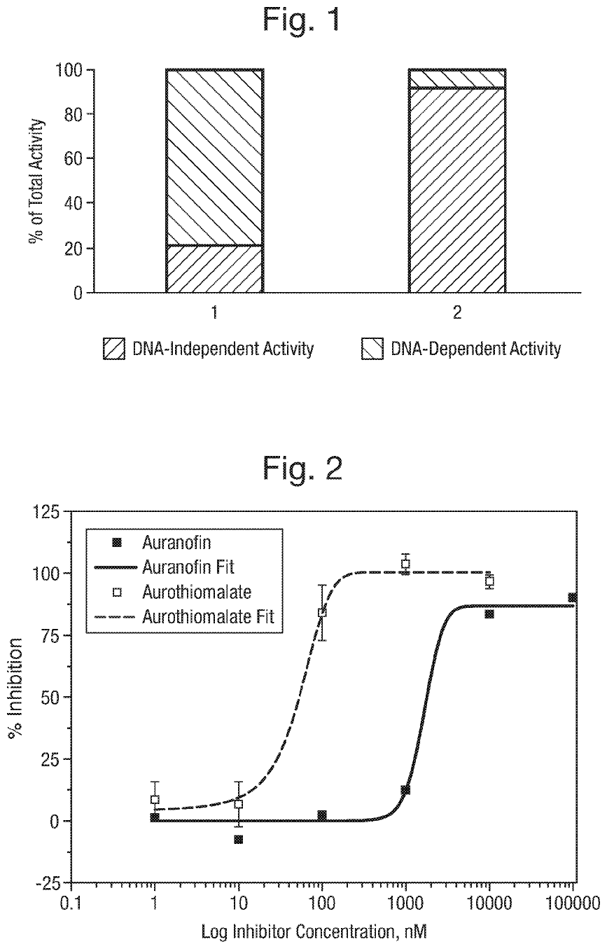

FIG. 1 is a graph showing how PARP1 and PARP2 activity is split between DNA-dependent and DNA-independent reactions;

FIG. 2 is a graph showing the percentage inhibition of PARP1 for different concentrations of auranofin and aurothiomalate;

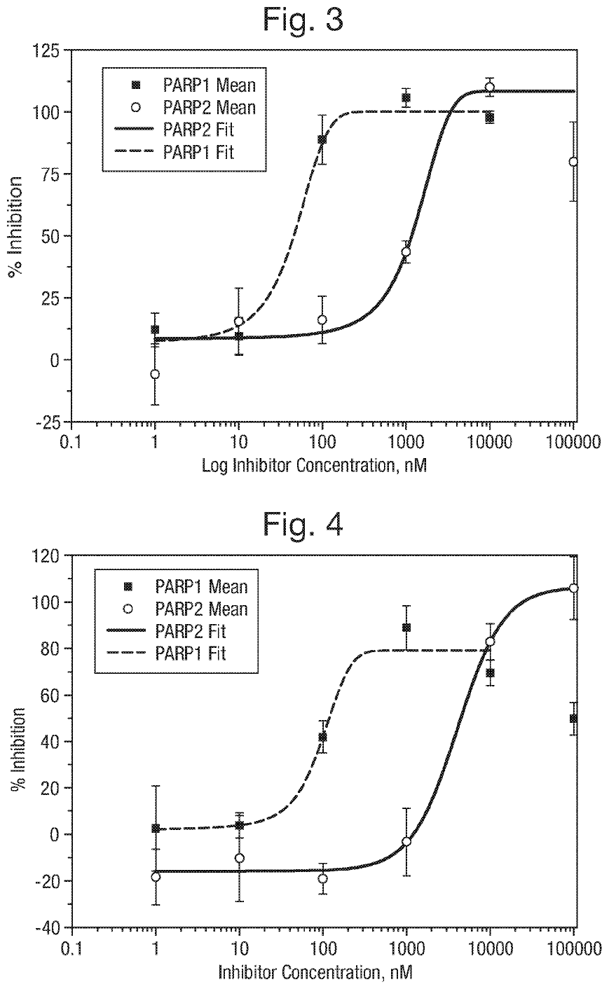

FIG. 3 is a graph showing the percentage inhibition of PARP1 and PARP2 for different concentrations of aurothiomalate;

FIG. 4 is a graph showing the percentage inhibition of PARP1 and PARP2 for different concentrations of aurothioglucose;

FIG. 5 is a PARP amino acid sequence alignment;

FIG. 6 is a graph showing the percentage inhibition of PARP1 and PARP2 for different concentrations of minocycline;

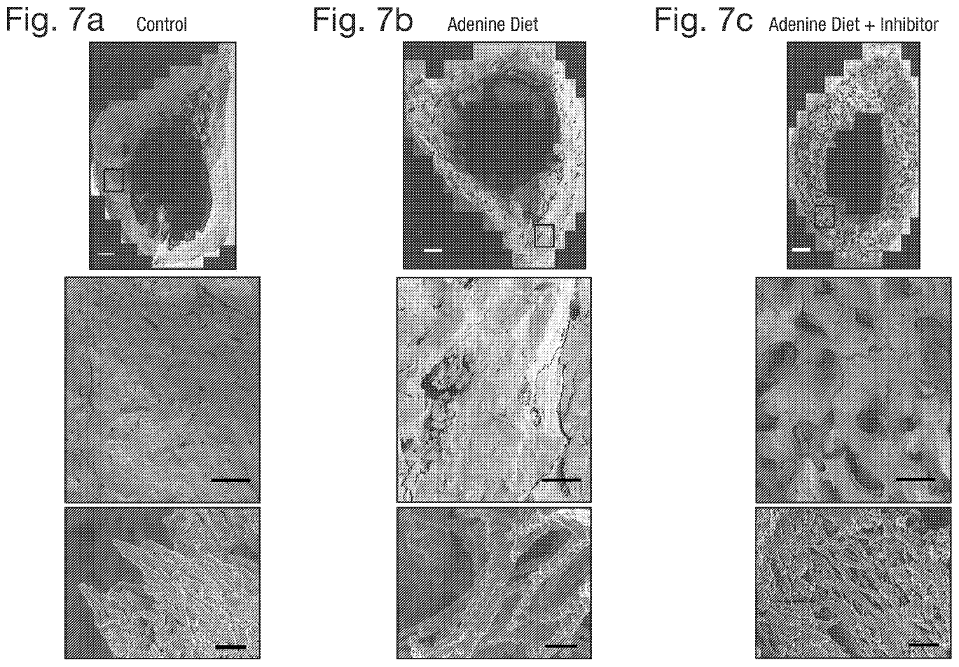

FIG. 7 shows scanning electron microscopy (SEM) and transmission electron microscopy (TEM) images of cross-sections of the long limb bone from rats where the rats were (a) untreated; (b) fed a high adenine/low protein diet which caused chronic kidney disease (CKD); or (c) fed a high adenine/low protein diet which caused CKD and administered minocycline; and

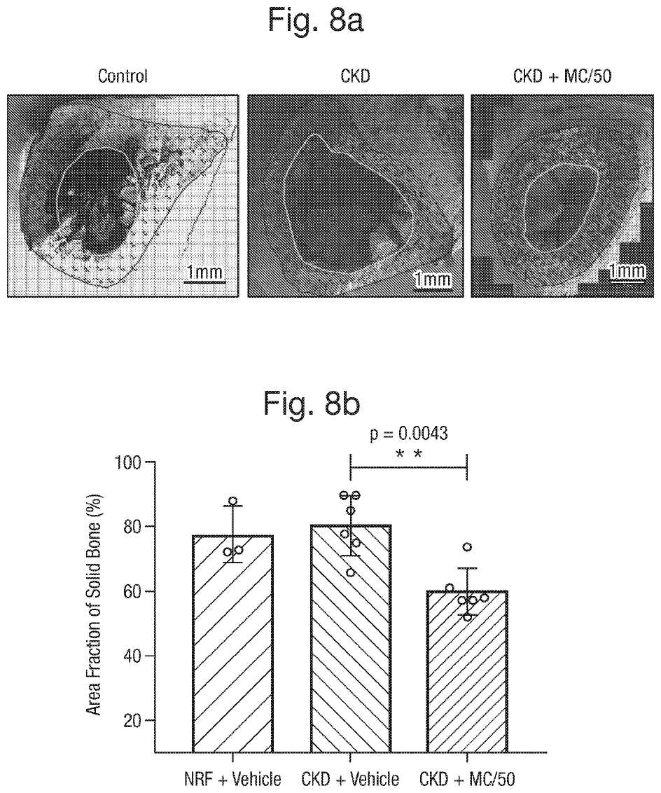

FIG. 8 shows analysis of the bone density of the long limb bone in the rats.

### EXAMPLE 1—ASSAYING OF DNA-DEPENDENT AND DNA-INDEPENDENT PARP1 ACTIVITY AND INHIBITOR DOSE-RESPONSES

The PARP inhibitor assay is a direct fluorescence-based concentration measurement of reaction product formation. The assay reagents are sold as a commercial kit (see http://www.merckmillipore.com/GB/en/product/PARP1-Enzyme-Activity-Assay,MM_NF-17-10149). To measure PARP inhibition, the NAD+ substrate concentration should be set at Km (the Michaelis constant) to enable identifications of all types of inhibitors (competitive, uncompetitive and non-competitive (allosteric) (the latter represents a mode of action of Zn-finger inhibitors)), direct calculation of inhibitor potency (Ki) and in vivo modelling. (See in and literature sited therein: Michael G. Acker, Douglas S. Auld. Considerations for the design and reporting of enzyme assays in high-throughput screening applications. Perspectives in Science (2014) 1, 56-73). All other PARP inhibitor assays reported in the literature (and including those available commercially) either alter NAD+ significantly to label it for measurement, or only include very small concentrations of NAD+(if at all) such that the competitive kinetics are not representative.

PARP activity and inhibition was measured for human full length active PARP1 (CS207770, Merck), PARP2 (ab198766, Abeam) and PARP3 (ab79638, Abeam) proteins. Inhibitor compounds (Sodium Aurothiomalate and Aurothioglucose, Sigma-Aldrich and Auranofin, Bio-Techne) at different concentrations (1, 10 and 100 nM, 1, 10 and 100 μM final) were added to the reaction buffer, concocted as a 1:1 mixture of Merck kit buffer with 50 mM Tris-HCl, 100 mM NaCl, 5 mM MgCl₂, 0.05% Tween-20, pH 8.0, Sigma), and incubated with PARP1 (2.5 ng/μL final), PARP2 (2.2 ng/μL final) or PARP3 (55 ng/μL final) at room temperature for 30 min.

Further, activated DNA (2 ng/μL final), 13-NAD (60 and 400 μM final for PARP1/2 and PARP3, respectively) and Nicotinamidase (200 ng/μL final) were added and incubated at 37° C. for 45 min. Total reaction volume was 25 μL.

Controls were executed as follows:
1. Control of 0% inhibition contained reaction sample without inhibitor;
2. Control of 100% inhibition of PARP1/2/3 activity contained reaction sample without β-NAD; and
3. Control of 100% inhibition of DNA-dependent activity contained reaction sample without DNA.

After the plates were cooled down to room temperature, 25 μL of Merck proprietary reagent was added to the reaction mixture and incubated at mild shaking for 45 min. Fluorescence measurement was carried out at excitation wavelength of 410 nm and emission of 460 nm in Fluostar Omega microplate reader (BMG Labtech).

Calculation of PARP1/2/3 Activity

Total PARP1/2/3 activity was calculated as a difference between control (1) and control (2). DNA-independent activity was calculated as a difference between control (1) and control (3). DNA-dependent activity was calculated as the difference between the total PARP1/2/3 activity and DNA-independent activity. As shown in FIG. 1, about 80% of PARP1 activity is DNA-dependent. However, potentially up to 30% of PARP1 activity can be DNA-independent.

Calculation of PARP inhibition

Inhibitory values were converted into percentages according to controls. Controls (1) and (3) were used in the case of PARP1, because only inhibition of DNA-dependent activity was observed, and the results are shown in FIG. 2. Controls (1) and (2) were used in the case of PARP2/3, because inhibition of total PARP2/3 activity (both DNA-dependent and DNA-independent reactions) was observed. FIGS. 3 and 4 show the percentage inhibition of PARP1 and PARP2 for different concentrations of aurothiomalate and aurothioglucose, respectively.

IC50 values were determined as inhibitor concentration at 50% inhibition, and are given in table 1.

TABLE 1
IC₅₀ values for Auranofin, Aurothiomalate and Aurothioglucose

| | IC₅₀/nm | | |
|---|---|---|---|
| | | Sodium | |
| | Auranofin | Aurothiomalate | Aurothioglucose |
| PARP1 | 1400 ± 98 | 48 ± 7 | 120 ± 7 |
| PARP2 | 1160 ± 81 | 1190 ± 190 | 4452 ± 623 |
| PARP3 | — | 1160 ± 93 | 1140 ± 20 |

As shown in FIG. 2 and Table 1, auranofin, as a mixed group aurothio- and phosphine compound only inhibits PARP1 and PARP2 at very high concentrations. Accordingly, auranofin is not suitable as a drug candidate, as doses this high are not known to be safe.

However, sodium aurothiomalate and aurothioglucose, i.e. pure aurothio compounds, have an IC₅₀ for PARP1 which is 30×-10× more potent than auranofin, so both are within acceptable safety dosage. Furthermore, as shown in FIGS. 3 and 4 and Table 1, neither aurothiomalate nor aurothioglucose inhibit PARP2 or PARP3, and so can be viewed as selective PARP1 inhibitors.

### EXAMPLE 2—EFFECT OF PARP2 INHIBITION ON BONE DENSITY

In order to prove that inhibiting PARP2 is a significant risk factor for osteoporosis, we first identified a PARP2-specific inhibitor, using the PARP inhibitor assay described in Example 1. Using this assay, the inventors found that minocycline is a specific PARP2 inhibitor and inhibits PARP2 with an IC₅₀ of 2.8 μM, and inhibits PARP1 with an IC₅₀ of 204.5 μM, see FIG. 6. It will be noted that the PARP2 vs PARP1 selectivity factor for minocycline is greater than 70×.

The effects of minocycline on bone calcification processes were evaluated in an in vivo rat model. The rats were fed a high adenine/low protein diet in order to develop chronic kidney disease (CKD) and associated hyperphosphatemia and medial vascular calcification. It is also expected to cause increased rates of bone turnover, allowing the inventors to examine whether inhibition of PARP2 enzymatic activity during bone remodelling affected mineralization.

14 of the rats on the high adenine/low protein diet were treated with 50 mg/kg/day of minocycline for 6 weeks. At the end of the study period cross sections of the long limb bone were analysed using scanning electron microscopy (SEM) and transmission electron microscopy (TEM), see FIG. 7, and the area fraction of solid bone in the cortical area of the bone cross section was quantified from these images, see FIG. 8. Statistical significance was determined by Mann-Whitney test.

As shown in FIG. 8b, a 25% reduction in the area fraction of solid bone was observed in the rats treated with the minocycline when compared to both the control and the rats which had been fed the high adenine/low protein diet but not treated with minocycline.

### CONCLUSIONS

The inventors believe that the reason sodium aurothiomalate and aurothioglucose inhibit PARP1 and not PARP2/3 is because they inhibit the PARP1 Zn finger domain/domains from binding to DNA, a pre-requisite step in the activation of PARP1 in DNA repair. It is thought that the Zn²⁺ ion is released and replaced by a Au⁺ ion and there is a conformational change. The resultant “gold finger” domain does not bind to DNA and therefore SSBs are not repaired. Accordingly, the synthetic lethality mechanism aimed at killing cancer cells is preserved.

The inventors have shown that PARP1 has DNA-independent activity. This activity is maintained in the presence of sodium aurothiomalate and aurothioglucose. Thus, PARP1 is available to undertake its other essential cellular DNA-independent roles in non-cancerous cells in the rest of the body.

The inventors have shown that PARP2 inhibition affects osteoblast function. Such inhibition would be particularly problematic in a patient suffering from or at increased risk of osteoporosis, e.g. a patient suffering from breast cancer or prostate cancer. Inhibition of osteoblast function would also be problematic, and greatly increase the risk of osteoporosis, in patients requiring long-term treatments, such as patients receiving maintenance therapy.

Furthermore, PARP2/3 activity is not inhibited by aurothiomalate and aurothioglucose, thus both enzymes are preserved to undertake their essential cellular roles, and osteoblast function will not be affected. Accordingly, the inventors have shown that aurothio compounds, such as aurothiomalate and aurothioglucose, could be used as highly selective oncology drugs for cancer therapy and/or as a second line of treatment to reduce drug resistance to other PARP inhibitors that target the catalytic site of PARP enzymes. This will be particularly beneficial for patients suffering from or at risk of osteoporosis. It will be noted that these compounds offer a significant advantage over approved drugs such as olaparib (LYNPARZA™) which inhibit both PARP1 and PARP2.

1. A method of treating, preventing or ameliorating cancer in a subject, the method comprising administering to a subject in need of such treatment, a therapeutically effective amount of a selective inhibitor of DNA-binding to poly (ADP-ribose) polymerase 1 (PARP1), or a pharmaceutically acceptable salt or solvate thereof, wherein the subject is suffering from or at risk of osteoporosis or requires a long-term therapy.

2. The method of claim 1, wherein the inhibitor does not inhibit the other functions of PARP1 besides DNA-binding.

3. The method of claim 2, wherein the other functions of PARP1 comprise PARP1's role in a cellular response to oxidative stress independent of DNA damage and/or PARP1's role in cell metabolic regulation and metabolic activity, calcium signalling and calcification, and apoptosis.

4. The method of claim 1, wherein the inhibitor does not inhibit or block the NAD+ binding site of PARP1.

5. The method of claim 1, wherein the inhibitor is an inhibitor of the zinc finger of PARP1.

6. The method of claim 1, wherein the subject is a post-menopausal woman, a woman who has had a hysterectomy before the age of 45, a woman who has suffered from absent periods for more than 6 months as a result of over exercising or too much dieting or a man suffering from hypogonadism.

7. The method of claim 1, wherein the subject is suffering from rheumatoid arthritis.

8. The method of claim 1, wherein the cancer is a solid tumour or solid cancer.

9. The method of claim 1, wherein the cancer is blood cancer, bowel cancer, brain cancer, breast cancer, cervical cancer, endometrial cancer, gastric cancer, liver cancer, lung cancer, ovarian cancer, pancreatic cancer, prostate cancer or skin cancer.

10. The method of claim 9, wherein the cancer is breast cancer, prostate cancer, myeloma or cervical cancer.

11. The method of claim 1, wherein the long-term therapy is maintenance therapy.

12. The method of claim 1, wherein the inhibitor is not an inhibitor of PARP2 and/or PARP3.

13. The method of claim 1, wherein the inhibitor is a gold complex, optionally a gold (I) complex.

14. (canceled)

15. The method of claim 1, wherein the inhibitor is a polymeric water-soluble complex.

16. The method of claim 1, wherein the inhibitor is a compound of Formula I, Formula II, Formula III, Formula IV or Formula V: 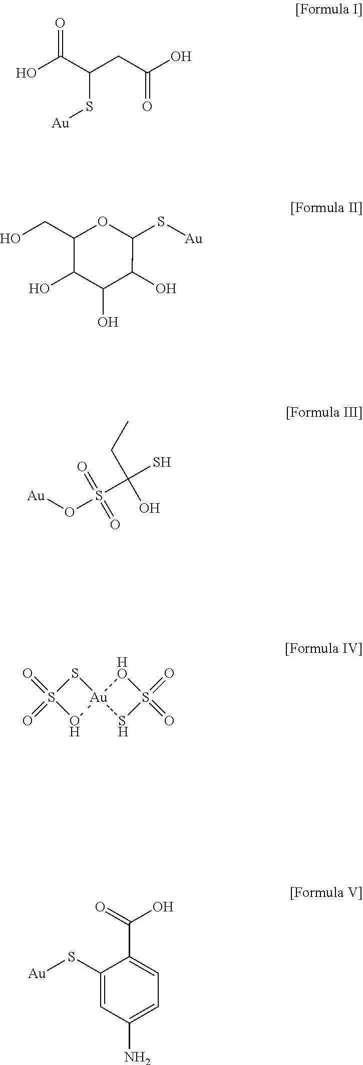
or a pharmaceutically acceptable salt and/or solvate thereof.

17. The method of claim 16, wherein the compound is a compound of Formula I or Formula II.

18. The method of claim 17, wherein the compound is a compound of Formula IIa: 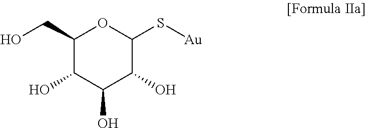
or a pharmaceutically acceptable salt and/or solvate thereof.

19. The method of claim 17, wherein the inhibitor is sodium aurothiomalate, potassium aurothiomalate or calcium aurothiomalate, optionally wherein the inhibitor is a compound of Formula Ia:

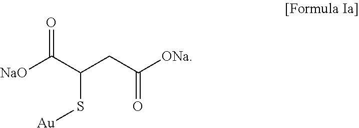

20. (canceled)

21. The method of claim 1, wherein the inhibitor is used in combination with a drug that damages DNA.

22. The method of claim 21, wherein the inhibitor is used in combination with an ataxia-telangiectasia mutated and rad3-related protein kinase (ATR) inhibitor, a checkpoint inhibitor, a vascular endothelial growth factor (VEGF) inhibitor or a wee1 inhibitor, optionally wherein the checkpoint inhibitor is a programmed cell death protein 1 (PD-1) inhibitor, a programmed death-ligand 1 (PD-L1) inhibitor or a cytotoxic T-lymphocyte-associated protein 4 (CTLA-4) inhibitor.

23-25. (canceled)
